(19) United States
(12) Patent Application Publication
(10) Pub. No.: US 2022/0071572 A1
(43) Pub. Date: Mar. 10, 2022

(54) METHOD AND APPARATUS FOR DISPLAYING OPERATION OF IMAGE POSITIONING, ELECTRONIC DEVICE, AND STORAGE MEDIUM

(71) Applicant: BEIJING SENSETIME TECHNOLOGY DEVELOPMENT CO., LTD., Beijing (CN)

(72) Inventor: Liwei ZHANG, Beijing (CN)

(21) Appl. No.: 17/526,102

(22) Filed: Nov. 15, 2021

Related U.S. Application Data
(63) Continuation of application No. PCT/CN2020/100717, filed on Jul. 7, 2020.

(30) Foreign Application Priority Data
Nov. 29, 2019 (CN) 201911204653.0

(51) Int. Cl. A61B 5/00

(52) U.S. Cl. CPC A61B 5/7425; A61B 6/5223; A61B 5/7445

(57) ABSTRACT

A method and apparatus for displaying operation of image positioning, an electronic device, and a storage medium are provided. The method includes: in response to a selection operation for any one of a plurality of operation areas, obtaining an operation position; triggering a corresponding operation manner at the operation position; obtaining, according to a correspondence relationship for the operation position among the plurality of operation areas, a linkage display result, based on the operation manner, for the plurality of operation areas.

### CROSS-REFERENCE TO RELATED APPLICATIONS

This is a continuation application of International Patent Application No. PCT/CN2020/100717, filed on Jul. 7, 2020, which is based on and claims priority to Chinese Patent Application No. 201911204653.0, which is filed on Nov. 29, 2019. The entire contents of International Patent Application No. PCT/CN2020/100717 and Chinese Patent Application No. 201911204653.0 are incorporated herein by reference in their entirety.

### BACKGROUND

In the process of displaying a two dimensional (2D) plane and modeling a three dimensional (3D) stereo model, for the target object and the positioning point in different operation areas (2D display areas, or 3D display areas obtained by modeling 3D stereo model), it is necessary to feedback space positions of a target object and a positioning point in a plurality of operation areas to a user for viewing. In order to analyze by further considering the display result for the plurality of operation areas, it is needed to view contrastively for the plurality of operation areas. However, in the related art, the displaying manner of contrastive viewing for space position is not intuitive, so that the display feedback of the space position cannot be obtained by user in time.

### SUMMARY

The present disclosure relates to the technical field of space positioning, and in particular, to a method and an apparatus for displaying operation of image positioning, an electronic device, and a storage medium.

Embodiments of the present disclosure provide a method and an apparatus for displaying operation of image positioning, an electronic device, and a storage medium.

The technical solutions of embodiments of the present disclosure are implemented in the following manner.

Embodiments of the present disclosure provide a method for displaying operation of image positioning. The method includes the following operations. In response to a selection operation for any one of a plurality of operation areas, an operation position is obtained. A corresponding operation manner is triggered at the operation position. A linkage display result, based on the operation manner, for the plurality of operation areas is obtained according to a correspondence relationship for the operation position among the plurality of operation areas.

Embodiments of the present disclosure provide an apparatus for displaying operation of image positioning. The apparatus includes an operation response unit, a triggering unit and an operation display unit. The operation response unit is configured to obtain an operation position in response to a selection operation of any one of a plurality of operation areas. The triggering unit is configured to trigger a corresponding operation manner at the operation position. The operation display unit is configured to obtain, according to a correspondence relationship for the operation position among the plurality of operation areas, a linkage display result, based on the operation mode, for the plurality of operation areas.

Embodiments of the present disclosure provide an electronic device. The electronic device includes a processor and a memory configured to store processor-executable instructions. The processor is configured to perform the above method for displaying operation of image positioning.

Embodiments of the present disclosure provide a computer readable storage medium in which computer program instructions are stored, and when the computer program instructions are executed by a processor, the above method for displaying operation of image positioning is implemented.

It should be understood that the above general description and the following detailed description are exemplary and explanatory only and are not intended to limit the embodiments of the present disclosure.

Other features and aspects of the present disclosure are clear referring to the following detailed description of exemplary embodiments with reference to the accompanying drawings.

### BRIEF DESCRIPTION OF THE DRAWINGS

The accompanying drawings, which are incorporated in and constitute a part of the description, illustrate embodiments consistent with the present disclosure and, together with the description, serve to explain embodiments of the present disclosure.

FIG. 1 illustrates a flowchart of a method for displaying operation of image positioning according to embodiments of the present disclosure.

FIG. 2 illustrates operation display diagrams in transverse-coronal-sagittal views located at an orthogonal position for a plurality of operation areas according to embodiments of the present disclosure.

FIG. 3 illustrates operation display diagrams in transverse-coronal-sagittal views when moving to the right for a plurality of operation areas according to embodiments of the present disclosure.

FIG. 4 illustrates operation display diagrams in transverse-coronal-sagittal views in rotation for a plurality of operation areas according to embodiments of the present disclosure.

FIG. 5 illustrates an operational tool display diagram according to embodiments of the present disclosure.

FIG. 6 illustrates a block diagram of an apparatus for displaying operation of image positioning according to embodiments of the present disclosure.

FIG. 7 illustrates a block diagram of an electronic device according to embodiments of the present disclosure.

FIG. 8 illustrates a block diagram of an electronic device according to embodiments of the present disclosure.

### DETAILED DESCRIPTION

In the related art, the displaying manner of contrastive viewing for space position is not intuitive, so that the display feedback of the space position cannot be obtained by user in time.

According to the embodiments of the present disclosure, a method for displaying operation of image positioning is provided. A linkage display result may be obtained by performing linkage display for a plurality of operation areas according to a correspondence relationship for the operation position among the plurality of operation areas and a corresponding operation manner triggered at the operation position. According to the corresponding matching of the space positioning and the intuitive linkage display manner, which are beneficial to contrastive viewing for the plurality of operation areas in the analyzing process considering the display result for the plurality of operation areas additionally, the effect of feedback display is improved. And a next expected processing may be performed by the user in time according to the effect of feedback display. The interactive feedback speed is thus improved.

In a possible implementation, before the corresponding operation manner is triggered at the operation position, the method further includes the following operations. A position of the operation position relative to an operation area indication object is determined to obtain a determination result. The operation manner is determined according to the determination result.

According to the embodiments of the present disclosure, the position of the operation position relative to the operation area indication object is determined. And the operation manner is determined based on the obtained determination result, so as to obtain different operation manners by tracking different operation positions, thereby performing linkage display for the plurality of operation areas based on a current operation manner. It is beneficial to perform contrastive viewing for the plurality of operation areas in the analyzing process considering the display result for the plurality of operation areas additionally, and the effect of feedback display is improved.

In a possible implementation, after the corresponding operation manner is triggered at the operation position, the method further includes the following operation. In response to a position change of the operation position, the operation manner is switched to an operation manner after the position change. Different operation manners correspond to different operation tool display states respectively.

According to the embodiments of the present disclosure, for the position change of the operation position, the corresponding operation manner after the position change is switched. Therefore, different operation tool display states may correspond to different operation manners respectively, and the user may be assisted to perform the analysis processing in the process of contrastive viewing by further considering the display result for the plurality of operation areas, so as to improve the efficiency of the analysis processing.

In a possible implementation, the operation that in response to a position change of the operation position, the operation manner is switched to an operation manner after the position change includes the following operations. In response to the position change of the operation position, a first position after position change is obtained. In a case where the first position is located in a first preset area, the operation a second preset area, the operation manner is switched to a rotation operation.

According to the embodiments of the present disclosure, in a case where the operation position is changed to the first position, if the first position is located in the first preset area, the operation manner is switched to a moving operation. In a case where the operation position is changed to the second position, if the second position is located in the second preset area, the operation manner is switched to a rotation operation. Since the corresponding operation manner may be switched by tracking the change of the operation position, it is convenient for user to perform analyze in the process of contrastive viewing by further considering the display result for the plurality of operation areas, so as to improve the efficiency of the analysis processing.

In a possible implementation, the moving operation includes at least one of following moving operations: moving up, moving down, moving left, and moving right.

According to the embodiments of the present disclosure, a plurality of different manners of moving operations may be performed, thereby enriching the operation forms of viewing processing for the user.

In a possible implementation, the operation that a linkage display result is obtained based on the operation mode for the plurality of operation areas according to the correspondence relationship for the operation position among the plurality of operation areas includes the following operation. In a case where the plurality of operation areas represent a 2D image and a 3D image respectively, linkage processing is performed according to the correspondence relationship between a operation position in the 2D image and a operation position in the 3D image to obtain the linkage display result for the plurality of operation areas based on the operation manner.

According to the embodiments of the present disclosure, the linkage processing may be performed according to the correspondence relationship between an operation position in 2D image and an operation position in the 3D image based on the operation manner for the plurality of operation areas to obtain the linkage display result. It is convenient for the user to perform contrastive viewing by further considering the display result for the plurality of operation areas. The efficiency of viewing processing is thus improved.

In a possible implementation, after the operation that a linkage display result, based on the operation manner, for the plurality of operation areas is obtained according to the correspondence relationship for the operation position among the plurality of operation areas, the method further includes the operations. In response to a triggering operation of an operation list of any one of the plurality of operation areas, the operation list is obtained. An orthogonal position recovery operation is performed according to a target entry in the operation list. Linkage processing for the plurality of operation areas is performed based on the orthogonal position recovery operation to obtain an updated linkage display result.

According to the embodiments of the present disclosure, an operation list may be obtained through a triggering operation of an operation list of any one of the plurality of operation areas. Since an orthogonal position recovery operation may be directly triggered according to the operation list, and linkage processing may be performed based on the orthogonal position recovery operation to obtain the updated linkage display result, it is convenient for the user to perform contrastive viewing by further considering the display result for the plurality of operation areas. The efficiency of viewing processing is thus improved.

According to the embodiments of the present disclosure, in response to a selection operation of any one of a plurality of operation areas, an operation position is obtained. A corresponding operation manner is triggered at the operation position. A linkage display result, based on the operation manner, for the plurality of operation areas is obtained according to a correspondence relationship for the operation position among the plurality of operation areas. After the corresponding operation manner is triggered at the operation position, a linkage display result, based on the operation manner, for the plurality of operation areas may be obtained according to a correspondence relationship for the operation position among the plurality of operation areas. Therefore, a linkage display result may be obtained by performing linkage display for the plurality of operation areas according to the correspondence relationship for the operation position among the plurality of operation areas and the corresponding operation manner triggered at the operation position. According to the corresponding matching of the space positioning and the intuitive linkage display manner, which are beneficial to perform contrastive viewing for the plurality of operation areas in the analyzing process considering the display result for the plurality of operation areas additionally, the effect of feedback display is improved. And a next expected processing may be performed by the user in time according to the effect of feedback display. The interactive feedback speed is thus improved.

Various exemplary embodiments, features, and aspects of the present disclosure will be described in detail below with reference to the accompanying drawings. The same reference numerals in the drawings indicate identical or similar elements. Although various aspects of the embodiments are shown in the drawings, the drawings are not necessarily drawn to scale unless otherwise indicated.

The word “exemplary” as used herein means “serving as an example, an embodiment, or an illustration.” Any embodiments described herein as “exemplary” cannot be understood as superior or better to other embodiments.

The term “and/or,” as used herein, is merely an association relationship that describes an associated object, meaning that there may be three relationships. For example, A and/or B may represent three cases, namely A alone, both A and B, and B alone. Additionally, the term “at least one” used herein represents any one of a plurality and any combination of at least two of the plurality. For example, including at least one of A, B, and C may represents the inclusion of any one or more elements selected from the set consisting of A, B, and C.

In addition, in order to describe the embodiments of the present disclosure preferably, numerous details are set forth in the following description. It is understood by those skilled in the art that the embodiments of the present disclosure may be implemented without certain details. In some embodiments, methods, means, elements, and circuits that are well known to those skilled in the art have not been described in detail in order to highlight the spirit of the embodiments of the present disclosure.

FIG. 1 illustrates a flowchart of a method for displaying operation of image positioning according to embodiments of the present disclosure. The method is applicable to an apparatus for displaying operation of image positioning. For example, in a case where the apparatus is deployed in a terminal device or a server or other processing device for performing, the space positioning, the operation display processing, and the like may be performed. The terminal device may be a User Equipment (UE), a mobile device, a cellular telephone, a cordless telephone, a Personal Digital Assistant (PDA), a handheld device, a computing device, a vehicle equipment, a wearable device, or the like. In some possible implementations, the processing method may be implemented by a processor calling computer-readable instructions stored in a memory. As shown in FIG. 1, the method includes the following operations.

At operation S101, in response to a selection operation of any one of a plurality of operation areas, an operation position is obtained.

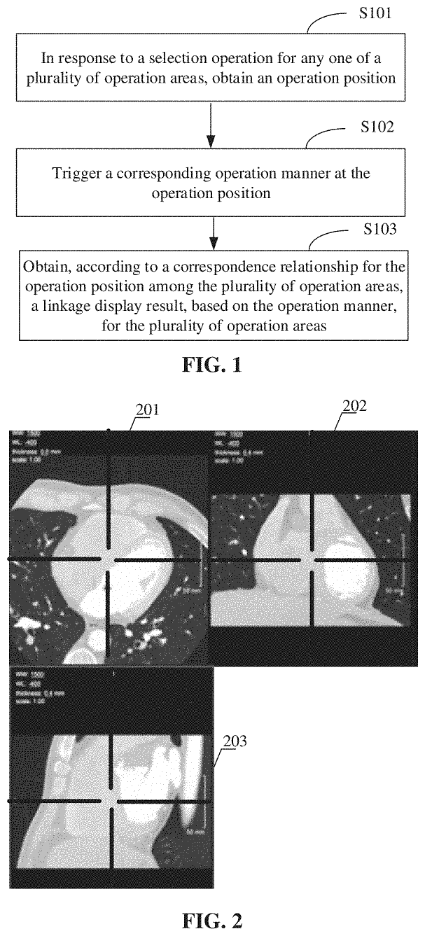

FIG. 2 illustrates an operation display diagrams in transverse-coronal-sagittal views located at an orthogonal position for a plurality of operation areas according to embodiments of the present disclosure. As shown in FIG. 2, the plurality of operation areas may include three operation areas, namely a transverse view, a coronal view and a sagittal view, which may be referred to as a transverse-coronal-sagittal views, and are identified by 201-203 respectively. The transverse view, which may be a 2D image, is displayed in the operation area 201. The coronal view, which may be a 3D reconstructed image, is displayed in the operation area 202. The sagittal view, which may be a 3D reconstructed image, is displayed in the operation area 203. The transverse view corresponds to a front view, the coronal view corresponds to a side view, and the sagittal view corresponds to a top view. FIG. 2 also includes an indication cross line. In addition to indicate the operation position, the current operation display interface may be changed by controlling the operation manners for moving or rotating the cross line. The selection operation may be a pointing operation, a clicking operation, a dragging operation, or the like to a position in any one of the plurality of operation areas. The certain position is the operating position.

At operation S102, a corresponding operation manner is triggered at the operation position.

The operation manner may include a moving operation, a rotating operation, and the like. The moving operation includes at least one of the following moving operations: moving up, moving down, moving left, and moving right.

In one example, as shown in FIG. 2, the current operation display interface, which is an orthogonal position in FIG. 2, may be changed by controlling the operation manners for moving or rotating the cross line.

At operation S103, a linkage display result, based on the operation manner for the plurality of operation areas, is obtained according to a correspondence relationship for the operation position among the plurality of operation areas.

Since there is a correspondence relationship, for any one operation position, among an original 2D image and a plurality of 3D reconstructed images in a process of space positioning and reconstruction, the corresponding positions of the operation position in the plurality of operation areas may be obtained according to the correspondence relationship. Therefore, according to the embodiments of present disclosure, the display results corresponding to the plurality of operation areas may be displayed respectively at the corresponding positions in the plurality of operation areas, and the position changes of the display results for the plurality of operation areas are displayed in linkage. For example, a circular center position in a 2D plane is a center position of a ball in a corresponding 3D space.

In one example, according to a correspondence relationship for the operation position among the plurality of operation areas, the corresponding operation manner may be triggered synchronously at the operation position of different operation areas, and the linkage display result based on the operation manner may be obtained according to the operation manner.

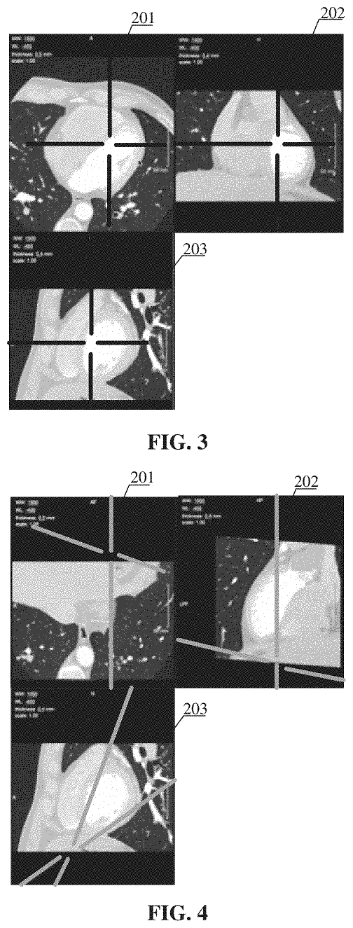

FIG. 3 illustrates operation display diagrams in transverse-coronal-sagittal views when moving to the right for a plurality of operation areas according to embodiments of the present disclosure. In the case where the operation of moving to the right is triggered at the operation position of one operation area (such as the operation area 201), the operation of moving to the right is triggered synchronously in the other two operation areas (such as the operation area 202 and/or the operation area 203). Therefore, the linkage display results of moving to the right are displayed in the transverse-coronal-sagittal views corresponding to the plurality of operation areas.

FIG. 4 illustrates operation display diagrams in transverse-coronal-sagittal views in rotation for a plurality of operation areas according to embodiments of the present disclosure. In a case where a rotation operation is triggered in an operation position of one operation area (such as the operation area 201), the rotation operation is also triggered synchronously in the other two operation areas (such as the operation area 202 and/or the operation area 203). Therefore, the linkage display results of rotation are displayed in the transverse-coronal-sagittal views corresponding to the plurality of operation areas.

That is, in the process of space positioning and reconstruction of the present embodiments of the present disclosure, the linkage display for the plurality of operation areas may be implemented according to the correspondence relationship of the plurality operation areas. That is, the operation manner triggered in any one operation area would lead to linkage processing for other operations areas to obtain the linkage display result.

According to the embodiments of the present disclosure, after a corresponding operation manner is triggered at the operation position, a linkage display result, based on the operation manner, for the plurality of operation areas may be obtained according to a correspondence relationship for the operation position among the plurality of operation areas. Therefore, a linkage display result may be obtained by performing linkage display for a plurality of operation areas according to a correspondence relationship for the operation position among plurality of operation areas. The corresponding matching of the space positioning and the intuitive linkage display manner are beneficial to perform contrastive viewing for the plurality of operation areas in the analyzing process considering the display result for the plurality of operation areas additionally. The next expected processing may be performed by the user in time according to the effect of display feedback, thereby improving the interactive feedback speed.

In a possible implementation manner, before the corresponding operation manner is triggered at the operation position, the method further includes the following operations. A position of the operation position relative to an operation area indication object is determined to obtain a determination result. The operation manner is determined according to the determination result.

In one example, the operation area indication object may be an indication cross line for indicating the horizontal-vertical direction in any operation area. The indication cross line as shown in FIGS. 2-4 may indicate the operation position, and change the current operation display interface by controlling the operation manners for moving or rotating the cross line. The determination result may include that the operation position is located in a middle region, for indicating the object, of the operation area, or that the operation position is located in an edge region, for indicating the object, of the operation area. Accordingly, in the case where the operation position is located in the middle region, for indicating the object, of the operation area, the operation manner may be a moving operation. In the case where the operation position is located in the edge region, for indicating the object, of the operation area, the operation manner may be a rotating operation.

In a possible implementation, after the corresponding operation manner is triggered at the operation position, the method further includes the following operation. In response to a position change of the operation position, the operation manner is switched to an operation manner after the position change. Different operation manners correspond to different operation tool display states respectively. That is, the corresponding operation manner may be displayed according to the position change. If the positions are different and the operation manners are different, the corresponding operation manners are different.

The position change of the operation position may be that a position of an operation position changes from a position located in a middle region of the operation area indication object to a position located in an edge region of the operation area indication object, and correspondingly, the operation manner is switched from a moving operation to a rotating operation. The operation tool display state corresponding to the rotating operation may be “half round”. The position change of the operation position may also be that a position of an operation position changes from a position located in an edge area of the operation area indication object to a position located in a middle region of the operation area indication object. Accordingly, the operation manner is switched from a rotating operation to a moving operation. The operation tool display state corresponding to the moving operation may be “cross”.

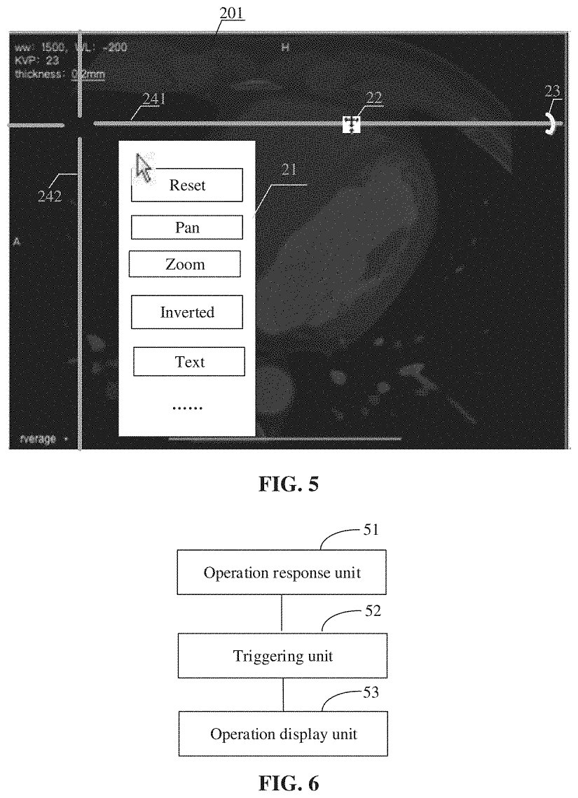

FIG. 5 illustrates an operational tool display diagram of according to embodiments of the present disclosure. For example, the above-mentioned indication cross line is included in the operation area 201. The cross line is composed of a first identification line 241 and a second identification line 242. An operation list 21 that may be triggered by a right mouse button, an operation tool pattern “cross” 22 for a moving operation, and an operation tool pattern “half round” for rotation are also included in the operation area 201. As shown in FIG. 5, the operation position is determined by the position of the mouse cursor on the cross line. In a case where the mouse cursor is located in the middle region of the first identification line 241 of the cross line, a moving operation, such as moving up, moving down, moving left or moving right, may be performed by the operation tool pattern “cross”. In a case where the mouse cursor is located in the edge region of the first identification line 241 of the cross line, the rotation operation may be performed by operating the tool pattern “half round”.

Whether the mouse cursor is located in the middle region or the edge region is determined according to a preset setting. For example, it may be set that if the position of the mouse cursor is located in 80% of the total length of the identification line (measured from the region edge), the mouse cursor is considered to be located in the middle region of one identification line of the cross line. If the position of the mouse cursor is located in 20% of the total length of the identification line (measured from the edge of the area), the mouse cursor is considered to be in the edge region of one identification line of the cross line.

According to the embodiments of present disclosure, the operation display may be recovered from a non-orthogonal position to the orthogonal position shown in FIG. 2, which may be implemented through the operation list displayed in FIG. 5 and choose an operation option, corresponding to an orthogonal position recovery, in the operation list. After a linkage display result, based on the operation manner, for the plurality of operation areas is obtained according to a correspondence relationship for the operation position among the plurality of operation areas, the method further includes the following operations. In response to a trigger operation of an operation list of any one of the plurality of operation areas, the operation list is obtained. An orthogonal position recovery operation is performed according to a target entry in the operation list. And linkage processing is performed based on the orthogonal position recovery operation for the plurality of operation areas to obtain an updated linkage display result.

The target entry may be a Reset entry for recovering the operation display from a non-orthogonal position to an orthogonal position shown in FIG. 2. And the operation list further includes entries, such as Pan, Zoom, Inverted, Text, and the like.

In view of above, according to the operation list and each operation tool pattern in the embodiments of the present disclosure, a corresponding operation process will be performed by directly clicking without the need to perform an additional switching processing before perform the next operation processing in a plurality of operation processing, so that the user operation is simplified and the interactive feedback speed is improved.

In a possible implementation manner, the operation that in response to the position change of the operation position, the operation manner is switched to an operation manner after the position change further includes the following operations. In response to the position change of the operation position, a first position after the position change is obtained. In a case where the first position is located in a first preset area (e.g., a middle area), the operation manner is switched to a moving operation (for example, at least one of following moving operations: moving up, moving down, moving left, and moving right). Alternatively, in a case where the first position is located in the second preset area (for example, the edge area), the operation manner is switched to a rotation operation (for example, a rotation operation with rotation angles such as 30 degrees, 45 degrees, and 60 degrees, or the like).

### Application Examples

In a scene of contrastive viewing for panes of transverse-coronal-sagittal views of the plurality operation areas, it is needed to position the position that the user needs to view, and the mouse cursor may be moved to the middle region of an identification line of the cross line. The position of mouse cursor will be changed into a tool of moving pattern, that is, an operation tool pattern “cross”, through which the moving operations such as moving up, moving down, moving left or moving right may be performed. The mouse cursor may be moved to edge regions of two sides of one identification line of the cross line, and the position of mouse cursor will be changed into a rotation pattern, that is, an operation tool pattern “half round”, through which a rotation operation may be performed, and the identification line is rotated to facilitate contrastively viewing any space slice. If any one operation manner (moving operation or rotating operation) is triggered, and the obtained operation interface display is a linkage display result, that is, any operation is linkage in transverse-coronal-sagittal views. If the user wants to recover to a vertical orthogonal position, the operation list may be called out, and the corresponding operation option for restoring to the orthogonal position may be selected to recover the orthogonal cross line to the orthogonal position.

In the related art, when viewing medical images, the operation is complicated. Sub-operations are required to motivate tools for different positions or different manners. Multiple times of switching tools are required to be adjusted back and forth. Multiple times of switching and manual adjustment are required. The user's understanding cost is increased, the operation efficiency is low, and user experience is affected.

In one embodiment of the present disclosure, in medical images, contents (similar to a front view, a side view, and a top view) of a three-dimensional space are composed by a set of original images and two sets of reconstructed images together. Because each set of data consists of multiple layers (similar to cutting an object into multiple slices and viewing the sections). The user needs to view the reconstructed image content through different sections and directions in multiple angles.

The embodiments of the present disclosure may be used to design a cross-line multi-planes reconstruction operation scheme. When an image is viewed in three panes of a transverse-coronal-sagittal views (a transverse view, a coronal view, and a sagittal view), page-turning, magnifying, and cross-line operations may be performed by a user on the image to locate a position to be viewed by the user. As shown in FIG. 5, the first identification line 241 and the second identification line 242 form a positioning cross-line, and the image may be moved up, moved down, moved left or moved right by the user through the middle region of each line. The mouse cursor may be moved to edge areas of two sides of the line, and the mouse cursor will be changed into a rotation pattern. The line may be rotated at this time, and any operation is linkage in the transverse-coronal-sagittal views, so that viewing of any space slice may be implemented. When the user wants to recover to the vertical position, as shown in FIG. 5, the menu may be called out by the right-click on the mouse. Recovering orthogonal cross line may be performed according to the Reset entry in the operation list.

According to the embodiments of the present disclosure, the determination of operation area may be implemented and switching may be performed in time. The operation display may be recovered from the non-orthogonal position to the orthogonal position by one operation using one key. There is no need for other activation operations, and real-time feedback may be provided. According to the scheme, the current operation manner may be determined in real time according to different image proportion position areas, the corresponding tool patterns may be switched in time, and multi-angle free positioning may be implemented.

Embodiments of the present disclosure may be applied to all logical operations having a correspondence relationship such as a reading system in department of radiology, an AI-assisted diagnosis, an AI labeling system, a telemedicine diagnosis, a cloud platform-assisted intelligent diagnosis and scanning workstations such as, Computed Tomography (CT), Magnetic Resonance (MR), positron emission tomography (PET), and the like.

It should be understood by those skilled in the art that in the above methods of the embodiments, the order in which the operations are described does not imply a strict order of execution that constitutes any limitation on the implementation process, and that the order in which the operations are executed should be determined in terms of their function and possible intrinsic logic.

Each of the above method embodiments mentioned in the present disclosure may be combined with each other to form a combined embodiment without departing from the principle logic. Due to the limit of space, details are not described herein for brevity.

In addition, the embodiments of the present disclosure further provides an apparatus for displaying operation of image positioning, an electronic device, a computer-readable storage medium, and a program. All the above can be used to implement any one of the method for displaying operation of image positioning provided in the embodiments of the present disclosure.

FIG. 6 illustrates a block diagram of an apparatus for displaying operation of image positioning according to embodiments of the present disclosure. As shown in FIG. 6, the e apparatus includes an operation response unit 51, a triggering unit 52 and an operation display unit 53. The operation response unit 51 is configured to obtain an operation position in response to a selection operation of any one of a plurality of operation areas. The triggering unit 52 is configured to trigger a corresponding operation manner at the operation position. The operation display unit 53 is configured to obtain, according to a correspondence relationship for the operation position among the plurality of operation areas, a linkage display result, based on the operation mode, for the plurality of operation areas.

In a possible implementation, the apparatus further includes a determining processing unit. The determining processing unit is configured to determine a position of the operation position relative to an operation area indication object to obtain a determination result, and determine the operation manner according to the determination result.

In a possible implementation, the apparatus further includes an operation switching unit. The operation switching unit is configured to switch the operation manner to an operation manner after position change in response to a position change of the operation position. Different operation manners correspond to different operation tool display states respectively.

In a possible implementation, the operation switching unit is configured to obtain a first position after the position change in response to the position change of the operation position, switch the operation manner to a moving operation in a case where the first position is located in a first preset area, switch the operation manner to a rotation operation in a case where the first position is located in the second preset area.

In a possible implementation, the moving operation includes at least one of following moving operations: moving up, moving down, moving left, and moving right.

In a possible implementation, the operation display unit is configured to: in a case where the plurality of operation areas represent a 2D image and a 3D image respectively, perform, according to the correspondence relationship between an operation position in the 2D image and an operation position in the 3D image, linkage processing, based on the operation manner, for the plurality of operation areas to obtain the linkage display result.

In a possible implementation, the apparatus further includes an orthogonal recovery unit. The orthogonal recovery unit is configured to: obtain the operation list in response to a triggering operation of an operation list of any one of the plurality of operation areas, perform an orthogonal position recovery operation according to a target entry in the operation list, and perform linkage processing based on the orthogonal position recovery operation for the plurality of operation areas to obtain an updated linkage display result.

In some embodiments, the apparatus provided by the embodiments of the present disclosure may have functions or include modules that may be configured to perform the methods described in the above method embodiments, the implementation of which may be understood with reference to the above method embodiments, and details are not described herein for brevity.

The embodiments of the present disclosure provide a computer readable storage medium having stored thereon computer program instructions that, when executed by a processor, a method for displaying operation of image positioning as described above is implemented. The computer-readable storage medium may be a volatile computer-readable storage medium or a non-volatile computer-readable storage medium.

The embodiments of the present disclosure also provide a computer program product comprising computer readable codes. When computer readable codes are run on a device, a processor in a device executes instructions configured to implement the method for displaying operation of image positioning as provided in any of the above embodiments.

The embodiments of the present disclosure also provide another computer program product, which is configured to store computer readable instructions. When the instructions are executed, the operation of a method of displaying operation of image positioning provided in any of the above embodiments.

The computer program product may be implemented in hardware, software, or a combination thereof. In one embodiment, the computer program product is reflected as a computer storage medium. And in another embodiment, the computer program product is reflected as a software product, such as a Software Development Kit (SDK) or the like.

The embodiments of the present disclosure further provide an electronic device. The electronic device includes a processor and a memory. The processor is configured to perform the above method. The memory is configured to store processor-executable instructions.

The electronic device may be provided as a terminal, a server, or other form of device.

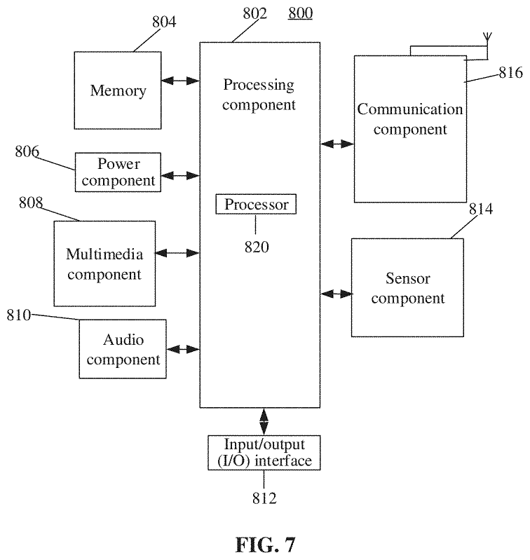

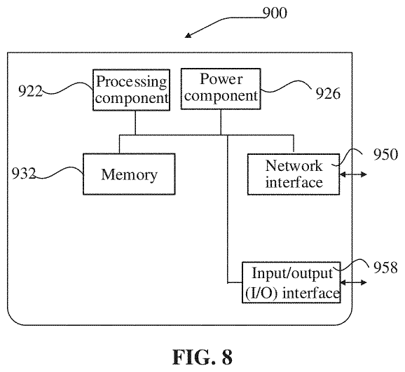

FIG. 7 illustrates a block diagram of an electronic device according to an embodiment of the present disclosure. For example, the electronic device 800 may be a mobile phone, a computer, a digital broadcast terminal, a messaging device, a game console, a tablet device, a medical device, an exercise device, a personal digital assistant, or the like.

Referring to FIG. 7, electronic device 800 may include one or more of the following: a processing component 802, a memory 804, a power component 806, a multimedia component 808, an audio component 810, an input/output (I/O) interface 812, a sensor component 814, and a communication component 816.

The processing component 802 generally controls the overall operation of the electronic device 800, such as operations associated with displays, telephone calls, data communications, camera operations, and recording operations. The processing component 802 may include one or more processors 820 to execute instructions to perform all or partial of the operations of the methods described above. In addition, the processing component 802 may include one or more modules to facilitate interaction between the processing component 802 and other components. For example, the processing component 802 may include a multimedia module to facilitate interaction between the multimedia component 808 and the processing component 802.

The memory 804 is configured to store various types of data to support operation at electronic device 800. Examples of such data include instructions of any application or method configured to operate on electronic device 800, such as, contact data, phone book data, messages, pictures, video, and the like. The memory 804 may be implemented by any type of volatile or non-volatile storage device or a combination thereof, such as Static Random-Access Memory (SRAM), Electrically Erasable Programmable read only memory (EEPROM), Erasable Programmable Read-Only Memory (EPROM), Programmable Read-Only Memory (PROM), Read-Only Memory (ROM), magnetic memory, flash memory, magnetic disk, or optical disk.

The power component 806 provides power to various components of electronic device 800. The power component 806 may include a power management system, one or more power supplies, and other components associated with generating, managing, and distributing power for electronic device 800.

The multimedia component 808 includes a screen providing an output interface between the electronic device 800 and a user. In some embodiments, the screen may include a Liquid Crystal Display (LCD) and a Touch panel (TP). In a case where the screen includes a touch panel, the screen may be implemented as a touch screen to receive input signals from a user. The touch panel includes one or more touch sensors to sense touch and slide, and gestures on the touch panel. The touch sensor may not only sense the boundary of a touch or sliding action, but also detect the duration and pressure associated with the touch or sliding operation. In some embodiments, the multimedia component 808 includes a front-facing camera and/or a rear-facing camera. In the case where the electronic device 800 is in an operation mode, such as a shooting mode or a video mode, the front-facing camera and/or the rear-facing camera may receive external multimedia data. Each of the front-facing camera and rear-facing camera may be a fixed optical lens system or have a focal length and optical zoom capability.

The audio component 810 is configured to output and/or input audio signals. For example, the audio component 810 includes a microphone (MIC). In a case where the electronic device 800 in an operating mode, such as a call mode, a recording mode, and a speech recognition mode, the microphone is configured to receive an external audio signal. The received audio signal may be stored in memory 804 or transmitted via communication component 816. In some embodiments, audio component 810 further includes a speaker configured to output an audio signal.

The I/O interface 812 provides an interface between the processing component 802 and a peripheral interface module. The above peripheral interface module may be a keyboard, a click wheel, a button, or the like. These buttons may include, but are not limited to, a home page button, a volume button, an activation button, and a lock button.

The sensor component 814 includes one or more sensors configured to provide various aspects of the state assessment for the electronic device 800. For example, the sensor component 814 may detect an on/off state of the electronic device 800, a relative positioning of the components, for example, the components are a display and keypad of the electronic device 800. The sensor component 814 may also detect a position change of the electronic device 800 or one of the components of the electronic device 800, whether there is contact between user and the electronic device 800, an orientation or acceleration/deceleration of the electronic device 800, and a temperature change in the electronic device 800. The sensor component 814 may include a proximity sensor configured to detect the presence of a nearby object without any physical contact. The sensor component 814 may also include a photo sensor, such as a Complementary Metal Oxide Semiconductor (CMOS) or Charge-coupled Device (CCD) 0 image sensor, configured for use in imaging applications. In some embodiments, the sensor component 814 may also include an acceleration sensor, a gyroscope sensor, a magnetic sensor, a pressure sensor, or a temperature sensor.

The communication component 816 is configured to facilitate wired or wireless communication between electronic device 800 and other devices. The electronic device 800 may access a wireless network based on a communication standard, such as WiFi, 2-Generation wireless telephone technology (2G) or 3-Generation wireless telephone technology (3G), or a combination thereof. In one exemplary embodiment, the communication component 816 receives broadcast signals or broadcast-related information from an external broadcast management system via a broadcast channel. In one exemplary embodiment, the communication component 816 further includes a Near Field Communication (NFC) module to facilitate short-range communication. For example, the NFC module may be implemented based on Radio Frequency Identification (RFID) technology, Infrared Data Association (IrDA) technology, Ultra Wide Band (UWB) technology, Bluetooth (BT) technology, and other technologies.

In an exemplary embodiment, the electronic device 800 may be implemented by one or more Application Specific Integrated Circuit (ASIC), Digital Signal Processing (DSP), Digital signal processing device (DSPD), programmable logic device (PLD), Field Programmable Gate Array (FPGA), controllers, microcontrollers, microprocessors, or other electronic components. The electronic device 800 is configured to perform the above-described methods.

In an exemplary embodiment, a non-volatile computer-readable storage medium is also provided. For example, a memory 804 is included in the above non-volatile computer-readable storage medium. The above computer program instructions can be executable by a processor 820 of the electronic device 800 to perform the methods described above.

FIG. 8 illustrates a block diagram of an electronic device 900 according to an embodiment of the present disclosure. For example, electronic device 900 may be provided as a server. Referring to FIG. 8, electronic device 900 includes a processing component 922 that includes one or more processors, and memory resources represented by a memory 932, configured to store instructions executed by processing component 922, such as applications. The application stored in memory 932 may include one or more modules each corresponding to a set of instructions. In addition, processing component 922 is configured to execute instructions to perform the methods described above.

The electronic device 900 may also include a power component 926 configured to perform power management of the electronic device 900, a wired or wireless network interface 950 configured to connect the electronic device 900 to a network, and an input/output (I/O) interface 958. The electronic device 900 may operate an operating system stored in memory 932, such as Windows Server™, Mac OS X™, Unix™, Linux™, FreeBSD™, or the like.

In an exemplary embodiment, a non-volatile computer-readable storage medium is also provided. For example, a memory 932 including computer program instructions is included in above non-volatile computer-readable storage medium. The computer program instructions can be executable by a processing component 922 of the electronic device 900 to perform the methods described above.

The embodiments of the present disclosure may be systems, methods, and/or computer program products. The computer program product may include a computer readable storage medium containing computer readable program instructions thereon configured to cause a processor to implement various aspects of embodiments of the present disclosure.

The computer-readable storage medium may be a tangible device that may hold and store instructions for use by the instruction execution device. The computer-readable storage medium may be, for example, but not limited to, an electrical storage device, a magnetic storage device, an optical storage device, an electromagnetic storage device, a semiconductor storage device, or any suitable combination of the above. Examples (non-exhaustive list) of the computer-readable storage medium include a portable computer disk, a hard disk, a Random Access Memory (RAM), a read-only memory (ROM), an erasable programmable read-only memory (EPROM or flash memory), a static random access memory (SRAM), a Compact Disc Read-Only Memory (CD-ROM), a Digital Video Disc (DVD), a memory stick, a floppy disk, a mechanical encoding device, such as a punch card or in-recess bump structure on which instructions are stored, and any suitable combination of the above. As used herein, a computer-readable storage medium is not to be explained as an instantaneous signal itself, such as a radio wave or other freely propagating electromagnetic wave, an electromagnetic wave propagating through a waveguide or other transmission medium (e.g., an optical pulse through a fiber optic cable), or an electrical signal transmitted through a wire.

The computer readable program instructions described herein may be downloaded from a computer readable storage medium to various computing/processing devices, or via a network, such as the Internet, a local area network, a wide area network, and/or a wireless network, to an external computer or external storage device. The network may include copper transmission cables, fiber optic transmission, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium in the respective computing/processing device.

The computer program instructions for performing the operations of the embodiments of the present disclosure may be assembly instructions, Industry Standard Architecture (ISA) instructions, machine instructions, machine-related instructions, microcode, firmware instructions, state setting data, or source code or object code written in any combination include object-oriented programming languages such as Smalltalk, C++, and the like, and conventional procedural programming languages such as “C” language or similar programming languages. The computer readable program instructions may be executed entirely on the user computer, partly on the user computer, as a separate software package, partly on the user computer and partly on the remote computer, or entirely on the remote computer or server. In the case involving a remote computer, the remote computer may be connected to the user computer through any kind of network including a local area network (LAN) or a Wide Area Network (WAN), or may be connected to an external computer (e.g., connection via the Internet through an Internet service provider). In some embodiments, various aspects of embodiments of the present disclosure are implemented by personalizing an electronic circuit, such as a programmable logic circuit, a field programmable gate array (FPGA), or a programmable logic array (PLA), with the status information of the computer-readable program instructions.

Aspects of embodiments of the present disclosure are described herein with reference to flow charts and/or block diagrams of methods, apparatus (systems), and computer program products in accordance with embodiments of the present disclosure. It should be understood that each block of the flowcharts and/or block diagrams, and combinations of blocks in the flowcharts and/or block diagrams, may be implemented by computer readable program instructions.

The computer readable program instructions may be provided to a processor of a general purpose computer, special purpose computer, or other programmable data processing apparatus to produce a machine such that the instructions, In a case where executed by the processor of the computer or other programmable data processing apparatus, produce apparatus for implementing the functions/acts specified in the flowchart and/or block diagram block or blocks. The computer-readable program instructions may also be stored in a computer-readable storage medium. The instructions cause a computer, programmable data processing apparatus, and/or other device to operate in a particular manner. Thereby, the computer-readable medium having the instructions stored thereon includes a manufacture that includes instructions that implement various aspects of the functions/acts specified in the flowchart and/or block diagram block or blocks.

Computer readable program instructions may also be loaded onto a computer, other programmable data processing apparatus, or other device such that a series of operational steps are performed on the computer, other programmable data processing apparatus, or other device to produce a computer-implemented process. Therefore, the functions/actions specified in one or more of the flowcharts and/or block diagrams can be implemented by the instructions that executed on the computer, other programmable data processing apparatus, or other device.

The flowcharts and block diagrams in the drawings illustrate systems, methods, and computer program products and architectures, functions, and operations of possible implementations of various embodiments of the present disclosure. In this regard, each block in a flowchart or block diagram may represent a module, program segment, or part of an instruction. The module, program segment, or part of an instruction contains executable one or more instructions for implementing a specified logical function. In some alternative implementations, the functions noted in the blocks may also occur in an order different from that noted in the drawings. For example, two successive blocks may actually be executed substantially in parallel, and they may sometimes be executed in the reverse order, depending on the functionality involved. It is also noted that each block of the block diagrams and/or flowcharts, and combinations of blocks in the block diagrams and/or flowcharts, may be implemented with a dedicated hardware-based system that performs the specified functions or actions, or may be implemented with a combination of dedicated hardware and computer instructions.

Various embodiments of the present disclosure may be combined with each other without departing from the logic, the description of the various embodiments being focused, and reference may be made to the description of other embodiments for the description of the various embodiments.

The various embodiments of the present disclosure have been described above. The above description is illustrative, not exhaustive, and is not limited to the various embodiments disclosed. Many modifications and variations will be apparent to those of ordinary skill in the art without departing from the scope and spirit of the illustrated embodiments. The choice of terms used herein is intended to best explain the principles of the various embodiments, practical applications, or technical improvements to the technology in the marketplace, or to enable others of ordinary skill in the art to understand the various embodiments disclosed herein.

### INDUSTRIAL APPLICABILITY

In the present embodiments, a linkage display result may be obtained by performing linkage display for a plurality of operation areas according to a correspondence relationship between the plurality of operation areas and an operation position and a corresponding operation manner triggered at the operation position. By the corresponding matching of the space positioning and the intuitive linkage display manner, it is beneficial to perform contrastive viewing for the plurality of operation areas in the analyzing process considering the display result for the plurality of operation areas additionally. The effect of display feedback is improved. The next expected processing may be performed by the user in time according to the effect of feedback display. Therefore, the interactive feedback speed is improved.

1. A method for displaying operation of image positioning, comprising:
in response to a selection operation for any one of a plurality of operation areas, obtaining an operation position;
triggering a corresponding operation manner at the operation position; and
obtaining, according to a correspondence relationship for the operation position among the plurality of operation areas, a linkage display result, based on the operation manner, for the plurality of operation areas.

2. The method according to claim 1, wherein before triggering the corresponding operation manner at the operation position, the method further comprises:
determining a position of the operation position relative to an operation area indication object to obtain a determination result; and
determining the operation manner according to the determination result.

3. The method according to claim 1, wherein after triggering the corresponding operation manner at the operation position, the method further comprises:
in response to a position change of the operation position, switching the operation manner to an operation manner after the position change,
wherein different operation manners correspond to different operation tool display states respectively.

4. The method according to claim 3, wherein in response to the position change of the operation position, switching the operation manner to the operation manner after the position change comprises:
in response to the position change of the operation position, obtaining a first position after the position change;
in a case where the first position is located in a first preset area, switching the operation manner to a moving operation; and
in a case where the first position is located in a second preset area, switching the operation manner to a rotation operation.

5. The method according to claim 4, wherein the moving operation comprises at least one of following moving operations: moving up, moving down, moving left, and moving right.

6. The method according to claim 1, wherein obtaining, according to the correspondence relationship for the operation position among the plurality of operation areas, the linkage display result, based on the operation manner, for the plurality of operation areas comprises:
in a case where the plurality of operation areas represent a 2D image and a 3D image respectively, performing, according to a correspondence relationship between the operation position in the 2D image and the operation position in the 3D image, linkage processing for the plurality of operation areas based on the operation manner to obtain the linkage display result.

7. The method according to claim 6, wherein after obtaining, according to the correspondence relationship for the operation position among the plurality of operation areas, the linkage display result, based on the operation manner, for the plurality of operation areas, the method further comprises:
in response to a triggering operation of an operation list of any one of the plurality of operation areas, obtaining the operation list;
performing, according to a target entry in the operation list, an orthogonal position recovery operation; and
performing, based on the orthogonal position recovery operation, linkage processing for the plurality of operation areas to obtain an updated linkage display result.

8. An electronic device, comprising:
a processor;
a memory configured to store processor-executable instructions,
wherein the processor is configured to:
obtain an operation position in response to a selection operation for any one of a plurality of operation areas;
trigger a corresponding operation manner at the operation position; and
obtain, according to a correspondence relationship for the operation position among the plurality of operation areas, a linkage display result, based on the operation manner, for the plurality of operation areas.

9. The electronic device according to claim 8, wherein the processor is further configured to:
determine a position of the operation position relative to an operation area indication object to obtain a determination result; and
determine the operation manner according to the determination result.

10. The electronic device according to claim 8, wherein the processor is further configured to:
in response to a position change of the operation position, switch the operation manner to an operation manner after the position change,
wherein different operation manners correspond to different operation tool display states respectively.

11. The electronic device according to claim 10, wherein the processor is further configured to:
in response to the position change of the operation position, obtain a first position after the position change;
in a case where the first position is located in a first preset area, switch the operation manner to a moving operation; and
in a case where the first position is located in a second preset area, switch the operation manner to a rotation operation.

12. The electronic device according to claim 11, wherein the moving operation comprises at least one of following moving operations: moving up, moving down, moving left, and moving right.

13. The electronic device according to claim 8, wherein the processor is further configured to:
in a case where the plurality of operation areas represent a 2D image and a 3D image respectively, perform, according to a correspondence relationship between the operation position in the 2D image and the operation position in the 3D image, linkage processing for the plurality of operation areas based on the operation manner to obtain the linkage display result.

14. The electronic device according to claim 13, wherein the processor is further configured to:
in response to a triggering operation of an operation list of any one of the plurality of operation areas, obtain the operation list;
perform, according to a target entry in the operation list, an orthogonal position recovery operation; and
perform, based on the orthogonal position recovery operation, linkage processing for the plurality of operation areas to obtain an updated linkage display result.

15. A non-transitory computer readable storage medium which computer program instructions are stored and when the computer program instructions are executed by a processor, a method comprising following operations is implemented:
in response to a selection operation for any one of a plurality of operation areas, obtaining an operation position;
triggering a corresponding operation manner at the operation position; and
obtaining, according to a correspondence relationship for the operation position among the plurality of operation areas, a linkage display result, based on the operation manner, for the plurality of operation areas.

16. The non-transitory computer readable storage medium according to claim 15, wherein before triggering the corresponding operation manner at the operation position, the operations further comprises:
determining a position of the operation position relative to an operation area indication object to obtain a determination result; and
determining the operation manner according to the determination result.

17. The non-transitory computer readable storage medium according to claim 15, wherein after triggering the corresponding operation manner at the operation position, the operations further comprises:
in response to a position change of the operation position, switching the operation manner to an operation manner after the position change,
wherein different operation manners correspond to different operation tool display states respectively.

18. The non-transitory computer readable storage medium according to claim 17, wherein in response to the position change of the operation position, switching the operation manner to the operation manner after the position change comprises:
in response to the position change of the operation position, obtaining a first position after the position change;
in a case where the first position is located in a first preset area, switching the operation manner to a moving operation; and
in a case where the first position is located in a second preset area, switching the operation manner to a rotation operation.

19. The non-transitory computer readable storage medium according to claim 18, wherein the moving operation comprises at least one of following moving operations: moving up, moving down, moving left, and moving right.

20. The non-transitory computer readable storage medium according to claim 15, wherein obtaining, according to the correspondence relationship for the operation position among the plurality of operation areas, the linkage display result, based on the operation manner, for the plurality of operation areas comprises:
in a case where the plurality of operation areas represent a 2D image and a 3D image respectively, performing, according to a correspondence relationship between the operation position in the 2D image and the operation position in the 3D image, linkage processing for the plurality of operation areas based on the operation manner to obtain the linkage display result.
